(19) United States
(12) Patent Application Publication
(10) Pub. No.: US 2010/0285086 A1
(43) Pub. Date: Nov. 11, 2010

(54) Biomimetic Extracellular Matrices

(76) Inventors: Mark H. Lee, Philadelphia, PA (US); David Boettiger, Wynnewood, PA (US); Russel Composto, Philadelphia, PA (US)

Correspondence Address: Pearl Cohen Zedek Latzer, LLP, 1500 Broadway, 12th Floor, New York, NY, 10036, US

(21) Appl. No.: 12/682,232

(22) PCT Filed: Oct. 9, 2008
(86) PCT No.: PCT/US08/79395

§ 371 Date: Jul. 12, 2010

Related U.S. Application Data
(60) Provisional application No. 60/960,652, filed on Oct. 9, 2007.

(51) Int. Cl. A61F 2/00; C12M 3/00; C12N 5/07

(52) U.S. Cl. 424/423; 435/289.1; 435/397

(57) ABSTRACT

The present invention is directed to hydrogel compositions for biotechnology applications. Specifically, the invention provides hydrogels mimicking the ECM and uses thereof.

### FIELD OF THE INVENTION

The present invention provides hydrogels for biotechnology applications. Specifically, the invention provides hydrogels mimicking the ECM and uses thereof.

### BACKGROUND OF THE INVENTION

The biochemical and mechanical cues provided by artificial extracellular matrices (ECMs) have been shown to dictate the adhesive and phenotypic response of many types of anchorage dependent cells. Namely, parameters such as substrate compliance and ligand density have been shown to be critical in the adhesion, spreading, migration and focal adhesion and cytoskeletal assembly of fibroblastic and smooth muscle cells. The transduction of these factors into intracellular signals is accomplished by the mechanical links between integrin receptors and ECM ligands presented by the substrate.

Extracellular matrices made from synthetic polymer hydrogels are used extensively as tissue engineering scaffolds and recently have found use as cell culture platforms. The properties of these scaffolds mediate the initial interactions with proteins and cells and provide mechanical support as the cells deposit their own matrix and organize into tissues. Dextran hydrogels are excellent candidates for these applications because they combine the biocompatibility of natural matrices with facile manipulability of the matrix's physicochemical properties found with synthetic alternatives.

Current tissue culture systems are primarily based on rigid polystyrene substrates that may denature the conformation of proteins during the adsorption process due to the polystyrene surface properties, which may adversely affect intracellular signaling networks that determine cell type and function through both distortion of adsorbed proteins and its rigidity. The realization of the limits of the standard tissue culture substrates (modified polystyrene and glass) has long been apparent to developmental biologists who developed protein coatings composed largely of extracellular matrix proteins to permit specific cell phenotypes to survive and express differentiated phenotypes. An early example was the use of rat tail collagen to promote muscle development. In contrast, chondrocyte development required conditions that reduced adhesion to a solid substrate. It has been long understood that the maintenance of stem cell populations required very specific conditions in their microenvironment for maintenance. As our knowledge of biology and potential medical applications for specific cell types expand, the number of specific cell types that must be maintained or expanded in culture has, and will continue, to increase. Over the past 20 years there has been a vast expansion in our understanding of soluble factors, growth factors and hormones that can influence cell behavior. These factors can be purified and presented to cells as soluble proteins. However, in vivo many of these will bind to different components of the extracellular matrix and hence presentation will be modulated. Also, the past 20 years has seen the identification of many cell-cell and cell-matrix adhesion molecules and an increasing appreciation of the fact that the adhesive connections have effects on cell behavior equally as fundamental as the soluble factors.

### SUMMARY OF THE INVENTION

In one embodiment, the invention provides a composition comprising a copolymer comprised of a first and a second dextran macromonomer wherein each of said first and second dextran macromonomer comprises a different degree of glycidylmethacrylate (GMA) substitution.

In another embodiment, the invention provides a biomimetic hydrogel comprising a copolymer having a first and a second dextran macromonomer wherein each said dextran macromonomer comprises a different degree of glycidylmethacrylate (GMA) substitution; and crosslinking agent.

In one embodiment, the invention provides a three-dimensional tissue scaffold for supporting tissue on-growth, the scaffold comprising: a substrate immobilized hydrogel, wherein the hydrogel comprises a copolymer having a first and a second dextran macromonomer wherein each of said first and second dextran macromonomer comprises a different degree of glycidylmethacrylate (GMA) substitution; and at least one of a living cell, an ECM ligand, protein, peptide, transcript factor, cytokine, therapeutic agent, growth factor, encapsulated in the hydrogel or on its surface.

In another embodiment, the invention provides a method of modulating the amount and location of an ECM ligand attachment on the surface of a hydrogel comprising the step of: selectively modifying the aldehyde concentration and location at the surface of a hydrogel comprising a first and a second dextran macromonomer wherein each of said first and second dextran macromonomer comprises a different degree of glycidylmethacrylate (GMA) substitution; and covalently binding the ECM ligand to the aldehyde group at the surface of the hydrogel.

In one embodiment, the invention provides a method of culturing living cells in a hydrogel while maintaining their phenotypic structure, comprising the steps of: encapsulating the cells in a hydrogel comprising a copolymer comprised of a first and a second dextran macromonomer wherein each of said first and second dextran macromonomer comprises a different degree of glycidylmethacrylate (GMA) substitution; modulating the storage modulus of the hydrogel; covalently binding the hydrogel to an ECM ligand; and allowing the cells to grow.

In another embodiment, the invention provides a method of controlling cell culture proliferation on a hydrogel surface, comprising the steps of immobilizing a hydrogel comprising a copolymer of a first and a second dextran macromonomer wherein each of said first and second dextran macromonomers comprises a different degree of glycidylmethacrylate (GMA) substitution, onto a substrate; modulating the storage modulus of the immobilized hydrogel; functionalizing the surface of the hydrogel with an ECM ligand; and seeding the hydrogel with the cell culture whose proliferation is sought to be controlled, wherein below a threshold storage modulus, no cell proliferation will occur.

In one embodiment, the invention provides a method of encapsulating a cell culture into a dextran hydrogel, comprising the steps of: suspending the cells in a macromonomer solution, wherein the macromonomer solution comprises a first and a second dextran macromonomer wherein each of said first and second dextran macromonomer comprises a different degree of glycidylmethacrylate (GMA) substitution; creating a gel from the macromonomers; using a crosslinker copolymerizing the gel.

In another embodiment, the invention provides a method of forming a tissue, the method comprising: providing a three-dimensional tissue scaffold-implant for supporting tissue on-growth, the scaffold-implant comprising: a substrate immobilized hydrogel, wherein the hydrogel comprises a copolymer having a first and a second dextran macromonomer wherein each of said first and second dextran macromonomer comprises a different degree of glycidylmethacrylate (GMA) substitution; and at least one of a living cell, an ECM ligand, protein, peptide, transcript factor, cytokine, therapeutic agent, growth factor, encapsulated in the hydrogel or on its surface; covering the surfaces of the scaffold with living cells; and culturing the scaffold to grow the tissue.

### BRIEF DESCRIPTION OF THE DRAWINGS

FIG. 1. Depicts a mold design for hydrogel fabrication.

FIG. 2. Depicts a graph showing FTIR spectrum of unmodified and GMA-modified dextran (2A). FIG. 2B. depicts a graph showing the carbonyl region of the FTIR spectra for macromonomers of varying degrees of substitution (DS). The intensity ratios of the peaks at 1710 cm⁻¹ (GMA) to 1600 cm⁻¹ for unmodified dextran and macromonomers for DS 1/23 and DS 1/10 were 0.18, 0.64 and 0.77. This is indicative of the increased incorporation of functional methacrylate units to the hydroxyl groups of dextran.

FIG. 3. Depicts a graph showing the In situ rheometric measurements of hydrogel formation from Dex-GMA macromonomers.

FIG. 4. Depicts graphs showing the effect of reaction parameters on Dex-GMA macromonomer gelation: A) Macromonomer concentration; B) initiator concentration; C) catalyst concentration; D) oxygenation of gelling medium. The compliance was most sensitive to the macromonomer concentration and the initiator and catalyst concentration had negligible effects, despite affecting gelation kinetics. The amount of oxygen in the polymerization buffer had a significant effect at lower macromonomer concentrations.

FIG. 5. Depicts graphs showing the compliance of one-component (A) vs. two-component (B) hydrogels. Two-component gel refers to the use of mixtures of macromonomers of two different DS. Both macromonomers were used at 200 mg/ml concentration. Standard gelation conditions were used. The two-component hydrogels resulted in a more uniform control over compliance while keeping constant dextran wt %.

FIG. 6. Depicts a scheme showing the activation of glass coverslips using methacrylpropyltrimethoxysilane. The modification was verified by monitoring the increases in film thickness and in water contact angle values.(Lee et al. 1721-30) Macromonomers gelled to these activated substrates were more stable to mechanical manipulation and to extended immersion in aqueous buffers.

FIG. 7. Depicts graphs showing the force-displacement spectroscopy of (A) an ultrathin film of dextran prepared by the method of Miksa et al.(Miksa et al. 557-64) and (B) an immobilized 45 KPa dextran hydrogel. The measurements were made in PBS using a MAC Type IV Si—N cantilever of 0.05 N/m spring constant. FIG. 7A shows clear transitions in stiffness as the tip traveled from I) before surface contact to II) compression of the hydrated layer and III) bending of the cantilever. Comparing the slopes of region II of the dextran film and the hydrogel substrate, the data shows that hydrated thin films of dextran is approximately 100 times stiffer than even the stiffest 45 KPa hydrogel used in this work.

FIG. 8. Depicts immobilized compliant dextran gels. Synthesis & biochemical and mechanical analyses, substrate modification are provided in FIGS. 8A-8E.

FIG. 9. Depicts control over ECM Ligand functionalization. Fine control over both the amount and spatial location of ECM ligand attachment to dextran gels was possible at the material level through the use of selective periodate oxidation and a hydrazide-based crosslinker [A]. Periodate concentration was used to sensitively control the amount of aldehyde groups generated for subsequent attachment [B]. In order to selectively modify only the surface of the hydrogels [C], stamps made of UV-treated polydimethylsiloxane (PDMS) stamps were used to localize the periodate oxidation. After immersion in a solution of AlexaFluor 488-labeled fibronectin, the ligand concentration was selectively enhanced at the surface [D]. By comparison, the fully oxidized gel (by immersion) was homogeneously fluorescent and the non-oxidized hydrogels were homogeneously unmodified.

FIG. 10. Depicts a 2D cell culture with dextran hydrogels. The effect of hydrogel compliance on human aortic smooth muscle cell (HASMC) function was assessed by spreading [A] and live/dead [B] assays at 1 and 5 days. By day 5, the HASMCs on stiff dextran substrates organized into multi-cellular structures with significant stress fibers [C-nuclear staining with DAPI is in blue; F-actin staining with phalloidin is in red]. Cells in differentiation media for 4 days [D].

FIG. 11. Depicts cell recovery by biodegradation of dextran hydrogels. The conditions of the enzymatic degradation of dextran hydrogels by dextranase [A] were optimized for use with dextran hydrogels. By using a colorimetric assay to monitor the degradative products of the hydrogel after 16 h exposure, the enzyme load, pH and Mn²⁺ion concentration were the key parameters controlling this reaction.[B] There are two ways in which the biodegradation of dextran can be of use in cell culture: The release of monolayers of 2D cultured cells [C] and the release of cells from three dimensional cultures of cells [D] without disruption of the deposited matrix or of the functional multi-cellular units formed by the cells.

FIG. 12. Depicts 3D cell culture with dextran hydrogels: HT1080 cell encapsulation.

### DETAILED DESCRIPTION OF THE INVENTION

In one embodiment, provided herein are hydrogels for biotechnology applications. In another embodiment, provided herein are hydrogels mimicking the ECM and uses thereof.

In one embodiment, the present invention provides a composition comprising a first and a second dextran macromonomer wherein each of said first and second dextran macromonomer comprises a different degree of substitution. In another embodiment, the first macromonomer comprises a degree of substitution between about 1/100 to ½. In another embodiment, the first macromonomer comprises a degree of substitution between about 1/100 to 1/80. In another embodiment, the first macromonomer comprises a degree of substitution between about 1/80 to 1/60. In another embodiment, the first macromonomer comprises a degree of substitution between about 1/60 to 1/40. In another embodiment, the first macromonomer comprises a degree of substitution between about 1/40 to 1/20.

In another embodiment, the first macromonomer comprises a degree of substitution between about 1/30 to 1/20. In another embodiment, the first macromonomer comprises a degree of substitution between about 1/30 to 1/25. In another embodiment, the first macromonomer comprises a degree of substitution between about 1/25 to 1/20. In another embodiment, the first macromonomer comprises a degree of substitution between about 1/20 to 1/15. In another embodiment, the first macromonomer comprises a degree of substitution between about 1/15 to 1/10. In another embodiment, the first macromonomer comprises a degree of substitution between about 1/13 to 1/10. In another embodiment, the first macromonomer comprises a degree of substitution between about 1/10 to ⅛. In another embodiment, the first macromonomer comprises a degree of substitution between about ⅛ to ⅕. In another embodiment, the first macromonomer comprises a degree of substitution between about ⅕ to ½. In one embodiment, the degree of substitution is measured in percentage of available substitution sites. Accordingly and in one embodiment, the degree of substitution on the dextran macromonomer is between about 3 and about 10%. In another embodiment, the degree of substitution is between about 3 and 33%, or in another embodiment, between 3 and 5%, or in another embodiment, between 5 and 10%, or in another embodiment, between 10 and 15%, or in another embodiment, between 15 and 20%, or in another embodiment, between 20 and 25%, or in another embodiment, between 25 and 30%.

In another embodiment, the second macromonomer comprises a degree of substitution between about 1/100 to ½. In another embodiment, the second macromonomer comprises a degree of substitution between about 1/100 to 1/80. In another embodiment, the second macromonomer comprises a degree of substitution between about 1/80 to 1/60. In another embodiment, the second macromonomer comprises a degree of substitution between about 1/60 to 1/40. In another embodiment, the second macromonomer comprises a degree of substitution between about 1/40 to 1/20.

In another embodiment, the second macromonomer comprises a degree of substitution between about 1/30 to 1/20. In another embodiment, the second macromonomer comprises a degree of substitution between about 1/30 to 1/25. In another embodiment, the second macromonomer comprises a degree of substitution between about 1/25 to 1/20. In another embodiment, the second macromonomer comprises a degree of substitution between about 1/20 to 1/15. In another embodiment, the second macromonomer comprises a degree of substitution between about 1/15 to 1/10. In another embodiment, the second macromonomer comprises a degree of substitution between about 1/13 to 1/10. In another embodiment, the second macromonomer comprises a degree of substitution between about 1/10 to ⅛. In another embodiment, the second macromonomer comprises a degree of substitution between about ⅛ to ⅕. In another embodiment, the second macromonomer comprises a degree of substitution between about ⅕ to ½.

In one embodiment, the degree of substitution of the macromonomers used in the compositions described herein will depend on the desired mechanical properties of the hydrogels described herein. The skilled practitioner would readily recognize that the higher the degree of substitution, the higher will the cross-linking density be and the lower will the concentration of hydrogel necessary to achieve the elasticity needed to support the embedded cells and the accompanying ECM proteins. It should be noted, that although throughout the description, the term “elasticity” or “elastic” is used, both the elastic and viscous components are considered encompassed by the compositions of the invention.

In another embodiment, the substitution is a methacrylate-hydroxyl substitution, a carbonate-hydroxyl substitution, or a mixture thereof. In another embodiment, methacrylate is a glycidylmethacrylate. In another embodiment, the substitution is an acrylate methacryloyl. In another embodiment, the substitution is an acryloyl bromide methacrylic. In another embodiment, the substitution is an acryloyl chloride methacrylic. In another embodiment, the substitution is an acrylic anhydride methacrylic. In another embodiment, the substitution is an acrylic acid hydroxyethyl-methacrylate. In another embodiment, the substitution is any other form of acrylate or any known derivative thereof. of these compounds

In another embodiment, the composition further comprises an extra cellular matrix protein. In another embodiment, the composition further comprises an extra cellular matrix protein covalently bound to the dextran macromonomer. In another embodiment, the extra cellular matrix protein is laminin. In another embodiment, the extra cellular matrix protein is fibronectin. In another embodiment, the extra cellular matrix protein is collagen. In another embodiment, the extra cellular matrix protein is gelatin. In another embodiment, the extra cellular matrix protein is pronectin. In another embodiment, the extra cellular matrix protein is elastin. In another embodiment, the extra cellular matrix protein is fibrillin. In another embodiment, the extra cellular matrix protein is a proteoglycan.

In another embodiment, collagen is type I collagen. In another embodiment, collagen is type II collagen. In another embodiment, collagen is type III collagen. In another embodiment, collagen is type IV collagen. In another embodiment, collagen is type V collagen. In another embodiment, collagen is type VI collagen. In another embodiment, collagen is type VII collagen. In another embodiment, collagen is type VIII collagen. In another embodiment, collagen is type IX collagen. In another embodiment, collagen is type X collagen. In another embodiment, collagen is type XI collagen. In another embodiment, collagen is type XIII collagen. In another embodiment, collagen is any combination of the various collagen types.

In another embodiment, the proteoglycan is chondroitin. In another embodiment, the proteoglycan is dermatan sulfate. In another embodiment, the proteoglycan is chondroitin sulfate. In another embodiment, the proteoglycan is heparan sulfate. In another embodiment, the proteoglycan is heparin.

In one embodiment, the macromonomer used as part of the systems described herein, is hyaluronic acid macromonomer, in combination with, in another embodiment, with the substituted dextrans described herein. In one embodiment, hyaluronic acid (HA) exists in animal tissues and is biocompatible and biodegradable. In one embodiment, HA hydrogel is obtained by chemically modifying hyaluronic acid, crosslinking the modified hyaluronic acid by methods known in the art to form a three dimensional network structure, and incorporating an aqueous medium such as water into the network structure. The HA hydrogel shows in one embodiment, viscoelastic as well as viscous properties. Likewise and in another embodiment, the systems described herein, comprising at least two macromonomers having different degrees of substitutions comprise HA and low DS dextrans.

Cross linking the substituted macromonomers described herein is carried out by, in one embodiment introducing nucleophilic agent, or photocuring, radiation, and others in other independent embodiments of the compositions and methods described herein. In one embodiment, biodegradability of the systems described herein is optimized by adjusting the cross link density and the degree of substitution, without compromising the mechanical properties of the resulting gel.

In another embodiment, a method of making a biomimetic hydrogel comprising the step of mixing a first and a second dextran macromonomer wherein each of the dextran macromonomer comprises a different degree of substitution; and crosslinking the first and second dextran macromonomer is provided.

In one embodiment, the storage modulus of the macromonomer systems described herein is important for the ability of the hydrogels to support proliferation, and differentiation of the cells embedded therein. The physical properties of any gel can be expressed by dynamic viscoelastic characteristics such as storage modulus (G′), loss modulus (G″), tangent delta (tan δ=G″/G′) and the like. A high storage modulus and a low loss modulus indicate high elasticity, meaning a hard gel. Conversely, a high loss modulus and a low storage modulus means a gel that is liquid-like (i.e., characterized by a viscosity). Therefore, in one embodiment, an elastic gel will have 0° phase shift in response to oscillatory deformation, wherein the input shear stress (F/A) τ=τ₀ sin(ωt) yields deformation γ, which is γ=γ₀ sin(ωt−δ)=γ₀ [sin(ωt)cos δ−cos(ωt)sin δ], where sin(ωt)cos δ, defines the elastic component of the gel and cos(ωt)sin δ; defines the viscous component of the gel. In one embodiment, the gels described herein are viscoelastic gels whose deformation in response to applied stress follows Hook's Law, where τ=G*γ, where τ is the shear stress as described above and γ is the ratio between the displacement in the direction of the deformation and the thickness of the gel at the place the shear stress is applied. G* is the complex modulus, defined in one embodiment as G*=τ/γ=G′+iG″ describing the mechanical characteristic of the gels described herein. Accordingly and in another embodiment, G′, the storage modulus is defined as G*cos δ and wherein ω is the angular velocity the oscillating deformation is being performed at (2πf). In one embodiment, the complex modulus of the crosslinked hydrogels described herein is adjusted to be equal to the complex modulus of the ECM in the desired tissue sought to be modeled or mimicked.

In one embodiment, the rheological properties of the systems comprising the macromonomers described in the methods and compositions provided herein are used to maintain embedded stem cells in a quiescent state and triggering differentiation is done by external means, such as change in ionic strength or the introduction of various growth factors as described herein.

In another embodiment, the storage modulus (G′) of the biomimetic hydrogel is between about 400 Pa to 42 KPa. In another embodiment, the storage modulus (G′) of the biomimetic hydrogel is between about 50 Pa, to 200 Pa. In another embodiment, the storage modulus (G′) of the biomimetic hydrogel is between about 200 Pa to 400 Pa. In another embodiment, the storage modulus (G′) of the biomimetic hydrogel is between about 400 pa to 1000 pa. In another embodiment, the storage modulus (G′) of the biomimetic hydrogel is between about 500 pa to 2 KPa. In another embodiment, the storage modulus (G′) of the biomimetic hydrogel is between about 1000 pa to 3 KPa. In another embodiment, the storage modulus (G′) of the biomimetic hydrogel is between about 3 KPa to 8 KPa. In another embodiment, the storage modulus (G′) of the biomimetic hydrogel is between about 5 KPa to 10 KPa. In another embodiment, the storage modulus (G′) of the biomimetic hydrogel is between about 10 KPa to 20 KPa. In another embodiment, the storage modulus (G′) of the biomimetic hydrogel is between about 18 KPa to 33 KPa. In another embodiment, the storage modulus (G′) of the biomimetic hydrogel is between about 20 KPa to 40 KPa. In another embodiment, the storage modulus (G′) of the biomimetic hydrogel is between about 30 KPa to 42 KPa.

In another embodiment, methods of making the novel gels of this invention comprise the use of initiators or polymerization catalysts. In another embodiment, the methods of the invention provide that crosslinking comprises mixing a crosslinking catalyst with a dextran macromonomers. In another embodiment, the catalyst is N,N,N′,N′-tetramethylethylenediamine (TEMED). In another embodiment, the catalyst is diethylmethylaminediamine (DEMED). In another embodiment, the catalyst is 3-dimethylaminopropionitrile (DMAPN) In another embodiment, the catalyst is ammonium persulfate-metabisulfite.

In another embodiment, the methods of the invention comprise free radical generating initiators. In another embodiment, the methods of the invention comprise azocompounds. In another embodiment, the methods of the invention comprise azodiiosobutyronitrile. In another embodiment, the methods of the invention comprise azodiisobutyramide. In another embodiment, the methods of the invention comprise azobis (dimethylvaleronitrile). In another embodiment, the methods of the invention comprise azobis (methylbutyronitrile). In another embodiment, the methods of the invention comprise dimethyl, diethyl, or dibutylazobismethylvalerate. In another embodiment, the methods of the invention comprise similar reagents containing a N,N double bond attached to aliphatic carbon atoms, at least one of which is tertiary. In another embodiment, the methods of the invention provide that the amount and type of initiator is generally indicated by the nature and concentrations of the monomer and crosslinking agent used. In another embodiment, the cross-linking agent is the compound bisacrylamide methylether (BAME), used either alone as cross-linking agent or in combination with other cross-linkers.

In one embodiment, provided herein is a composition comprising a copolymer comprised of a first and a second dextran macromonomer wherein each of said first and second dextran macromonomer comprises a different degree of glycidylmethacrylate (GMA) substitution.

In another embodiment, provided herein is a biomimetic hydrogel comprising a copolymer having a first and a second dextran macromonomer wherein each said dextran macromonomer comprises a different degree of glycidylmethacrylate (GMA) substitution; and crosslinking agent.

In one embodiment, provided herein is a three-dimensional tissue scaffold for supporting tissue on-growth, the scaffold comprising: a substrate immobilized hydrogel, wherein the hydrogel comprises a copolymer having a first and a second dextran macromonomer wherein each of said first and second dextran macromonomer comprises a different degree of glycidylmethacrylate (GMA) substitution; and at least one of a living cell, an ECM ligand, protein, peptide, transcript factor, cytokine, therapeutic agent, growth factor, encapsulated in the hydrogel or on its surface.

In another embodiment, provided herein is a method of modulating the amount and location of an ECM ligand attachment on the surface of a hydrogel comprising the step of: selectively modifying the aldehyde concentration and location at the surface of a hydrogel comprising a first and a second dextran macromonomer wherein each of said first and second dextran macromonomer comprises a different degree of glycidylmethacrylate (GMA) substitution; and covalently binding the ECM ligand to the aldehyde group at the surface of the hydrogel.

In one embodiment, provided herein is a method of culturing living cells in a hydrogel while maintaining their phenotypic structure, comprising the steps of: encapsulating the cells in a hydrogel comprising a copolymer comprised of a first and a second dextran macromonomer wherein each of said first and second dextran macromonomer comprises a different degree of glycidylmethacrylate (GMA) substitution; modulating the storage modulus of the hydrogel; covalently binding the hydrogel to an ECM ligand; and allowing the cells to grow.

In another embodiment, provided herein is a method of controlling cell culture proliferation on a hydrogel surface, comprising the steps of immobilizing a hydrogel comprising a copolymer of a first and a second dextran macromonomer wherein each of said first and second dextran macromonomers comprises a different degree of glycidylmethacrylate (GMA) substitution, onto a substrate; modulating the storage modulus of the immobilized hydrogel; functionalizing the surface of the hydrogel with an ECM ligand; and seeding the hydrogel with the cell culture whose proliferation is sought to be controlled, wherein below a threshold storage modulus, no cell proliferation will occur.

In one embodiment, provided herein is a method of encapsulating a cell culture into a dextran hydrogel, comprising the steps of: suspending the cells in a macromonomer solution, wherein the macromonomer solution comprises a first and a second dextran macromonomer wherein each of said first and second dextran macromonomer comprises a different degree of glycidylmethacrylate (GMA) substitution; creating a gel from the macromonomers; using a crosslinker copolymerizing the gel.

In another embodiment, provided herein is a method of forming a tissue, the method comprising: providing a three-dimensional tissue scaffold-implant for supporting tissue on-growth, the scaffold-implant comprising: a substrate immobilized hydrogel, wherein the hydrogel comprises a copolymer having a first and a second dextran macromonomer wherein each of said first and second dextran macromonomer comprises a different degree of glycidylmethacrylate (GMA) substitution; and at least one of a living cell, an ECM ligand, protein, peptide, transcript factor, cytokine, therapeutic agent, growth factor, encapsulated in the hydrogel or on its surface; covering the surfaces of the scaffold with living cells; and culturing the scaffold to grow the tissue.

In another embodiment, the methods comprise the use of free radical-providing initiator systems. In another embodiment, the methods comprise the use of free radical-providing initiator systems. In another embodiment, the free radical-providing initiator is benzoyl peroxide. In another embodiment, the free radical-providing initiator is t-butyl-hydroperoxide. In another embodiment, the free radical-providing initiator is lauroyl peroxide. In another embodiment, the free radical-providing initiator is cumene hydroperoxide. In another embodiment, the free radical-providing initiator is tetralin peroxide. In another embodiment, the free radical-providing initiator is acetyl peroxide. In another embodiment, the free radical-providing initiator is caproyl peroxide. In another embodiment, the free radical-providing initiator is t-butylperbenzoate. In another embodiment, the free radical-providing initiator is t-butyldiperphthalate. In another embodiment, the free radical-providing initiator is methylethylketone peroxide. In another embodiment, the free radical-providing initiator is hydrogen peroxide-Fe²⁺-ascorbic acid. In another embodiment, the free radical-providing initiator is riboflavin-light. In another embodiment, the free radical-providing initiator is methylene blue-light. In another embodiment, the free radical-providing initiator is a persulfate.

In another embodiment, the methods further comprise mixing a crosslinking initiator such as but not limited to ammonium persulfate (APS) or riboflavin/light.

In another embodiment, the invention comprises a hydrogel produced by the methods of the invention. In another embodiment, the invention comprises a biomimetic hydrogel produced by the methods of the invention.

In another embodiment, the invention provides a hydrogel having thickness of about 0.01 to 10 mm. In another embodiment, the invention provides a hydrogel having thickness of about 0.01 to 0.1 mm. In another embodiment, the invention provides a hydrogel having thickness of about 0.1 to 0.3 mm. In another embodiment, the invention provides a hydrogel having thickness of about 0.2 to 0.6 mm. In another embodiment, the invention provides a hydrogel having thickness of about 0.5 to 0.7 mm. In another embodiment, the invention provides a hydrogel having thickness of about 0.6 to 1 mm. In another embodiment, the invention provides a hydrogel having thickness of about 0.8 to 1.2 mm. In another embodiment, the invention provides a hydrogel having thickness of about 1.2 to 1.5 mm. In another embodiment, the invention provides a hydrogel having thickness of about 1.5 to 3 mm. In another embodiment, the invention provides a hydrogel having thickness of about 2 to 5 mm. In another embodiment, the invention provides a hydrogel having thickness of about 3 to 8 mm. In another embodiment, the invention provides a hydrogel having thickness of about 6 to 10 mm.

In another embodiment, the invention provides a method of making a substrate for growing, differentiating, or proliferating a cell, comprising the step of immobilizing the composition of the invention onto a substrate, thereby making a substrate for growing, differentiating, or proliferating a cell. In another embodiment, the cell of the invention is a solitary cell. In another embodiment, the cell of the invention a cell culture. In another embodiment, the cell of the invention a cell line. In another embodiment, the cell of the invention a primary cell culture. In another embodiment, the cell of the invention comprises a tissue. In another embodiment, the cell of the invention an organ or a fraction of an organ. In another embodiment, the cell is a eukaryotic cell. In another embodiment, the cell is an animal cell. In another embodiment, the cell is a human cell. In another embodiment, the cell is a plant cell. In another embodiment, the cell is a prokaryotic. In another embodiment, the cell is a transformed cell. In another embodiment, the cell is a cancerous cell.

In another embodiment, the substrate allows growth of a cell. In another embodiment, the substrate is manipulated in order to test the ability of a cell to adapt to the manipulation. In another embodiment, the substrate is manipulated to contain a growth factor. In another embodiment, the substrate is manipulated to contain an immune modulator such as but not limited to interleukin-2. In another embodiment, the substrate is manipulated to contain a hormone. In another embodiment, the substrate is manipulated to contain a drug. In another embodiment, the substrate is manipulated to contain an anti-cancer drug. In another embodiment, the substrate is manipulated to contain a certain profile on ECM proteins. In another embodiment, the substrate is manipulated to contain a certain concentration on ECM protein. In another embodiment, the surface charge of the substrate is manipulated.

In another embodiment, the substrate used to immobilize the compositions described herein, using the methods provided herein comprises any biocompatible material known in the art. In one embodiment, the substrate comprises silicon, or chromium, glass, stainless steel, polystyrene, tantalum, titanium, carbon, calcium phosphate, quartz, ceramic or their combination in other discrete embodiments. In another embodiment, the substrates used herein comprise hydroxyapatite or, in one embodiment, the common polymers that turn up as biomaterials such as poly(dimethylsiloxane), PMMA, PVC, Teflon, PE or polyurethan in certain embodiments. In one embodiment, the substrate comprises a mixture of the various substances provided herein.

In another embodiment, the substrate comprises a polymer. In another embodiment, the substrate comprises a thermoplastic polymer. In another embodiment, the substrate comprises an unsaturated ethylen monomers including olefins such as polyethylene, polypropylene, polybutylene, and copolymers of ethylene with higher olefins such as alpha olefins containing 4 to 10 carbon atoms or vinyl acetate, etc. In another embodiment, the substrate comprises vinyls such as polyvinyl chloride; polyvinyl esters such as polyvinyl acetate. In another embodiment, the substrate comprises polystyrene. In another embodiment, the substrate comprises an acrylic homopolymers. In another embodiment, the substrate comprises an acrylic copolymers. In another embodiment, the substrate comprises alkyds. In another embodiment, the substrate comprises amino resins. In another embodiment, the substrate comprises epoxy resins. In another embodiment, the substrate comprises polyamides. In another embodiment, the substrate comprises polyurethanes. In another embodiment, the substrate comprises phenoxy resins. In another embodiment, the substrate comprises polysulfones.

In another embodiment, the substrate comprises polycarbonates. In another embodiment, the substrate comprises polyesters and/or chlorinated polyesters. In another embodiment, the substrate comprises polyethers. In another embodiment, the substrate comprises acetal resins. In another embodiment, the substrate comprises polyimides. In another embodiment, the substrate comprises polyoxyethylenes.

In another embodiment, polymers according to the present invention also include various rubbers and/or elastomers either natural or synthetic polymers based on copolymerization, grafting, or physical blending of various diene monomers with the above mentioned polymers, all as generally known in the art.

In another embodiment, the step of immobilizing comprises the steps of: (a) activating the substrate; (b) immersing the activated substrate in an agent capable of forming a covalent bond between the activated substrate and the composition thereby making a functionalized substrate; (c) drying the functionalized substrate, and (d) contacting the functionalized substrate with the composition.

In another embodiment, the agent capable of forming a covalent bond between the activated substrate and the composition is alkylsilane. In another embodiment, silane monomers of different chemical structures are hydrolyzed or condensed to form copolymers, terpolymers, and so forth. In another embodiment, the alkylsilanes are formed with at least two different monomers, but no limit is placed on how many different monomers may be utilized. In another embodiment, the invention provides different monomers in the alkylsilane copolymer backbone. In another embodiment, the invention provides that different monomers in the alkylsilane copolymer backbone result in a polymer having different functional groups in the comonomers. In another embodiment, the dual functionality of the copolymer can provide stronger coupling to the fillers and better compatibility to the base resin.

In another embodiment, one monomer is unsaturated such as a vinyl. In another embodiment, crosslinking function is provided in addition to the coupling and compatibility functions. In another embodiment, terpolymers are designed using precondensation to provide three different functions including coupling, crosslinking and compatibility with the polymer resin depending on the type of silane or silicon compound chosen, and the pendant functional groups that silane or silicon compounds have.

In another embodiment, the alkylsilane copolymers are commercially available. In another embodiment, the alkylsilane copolymers are prepared by processes known in the art. In another embodiment, copolymers are prepared from silanes having at least 2 hydrolyzable groups through hydrolysis and condensation reactions. In another embodiment, silanes with a single hydrolyzable group is utilized to endcap the copolymers.

In another embodiment, alkylsilane copolymers and terpolymers of the present invention are formed using a variety of combinations including, for instance, an alkylsilane with 2 or 3 hydrolyzable groups such as alkoxy, acetoxy, hydroxy, or halide (in particular chloride), co-condensed with at least one second silane having at least 2 hydrolyzable groups such as methacryloxypropylsilane or vinyltrialkoxysilane, any silicon compound having at least 2 hydrolyzable groups such as tetraethylsilicate or tetramethylsilicate, or a linear or cyclic organosilicon compound such as tetracyclodimethylsiloxane (D4).

In another embodiment, silane monomers comprise but are not limited to, alkyltrialkoxysilanes such as Silquest A-162 methyltriethoxysilane supplied by Crompton Corp. in Middlebury, Conn.; Silquest) A-1630 methyltrimethoxysilane; Silquest A-137 octyltriethoxysilane; and Silquest Y-11869 octadecyltriethoxysilane. In another embodiment, Silquesto A-137 octyltriethoxysilane is utilized.

In another embodiment, alkylsilane monomers comprise but are not limited to, butyltriethoxysilane, dodecyltriethoxysilane, octyltrimethoxysilane, octadecyltrimethoxysilane, butyltrimethoxysilane, dodecyltrimethoxysilane, and mixtures thereof.

In another embodiment, the alkylsilane copolymers comprise a plurality of hydrolyzable groups. In another embodiment, the alkylsilane copolymers comprise at least two different monomers in their backbone structure. In another embodiment, the alkylsilane copolymers comprise at least three different monomers in their backbone structure. In another embodiment, the alkylsilane copolymers comprise at least four different monomers in their backbone structure. In another embodiment, the alkylsilane copolymers comprise at least five different monomers in their backbone structure. In another embodiment, the alkylsilane copolymers comprise at least six different monomers in their backbone structure.

In another embodiment, the silane copolymers are characterized by the following general formula: R[SiR₁R₂O]m-[SiR₃R₄O]nR₅ where R and R₁ are hydrolyzable groups such as alkoxy, halogen, acetoxy, hydroxy, and so forth, or mixture thereof; R₂ is a nonhydrolyzable C₁-C₂₀, aliphatic, cycloaliphatic or aromatic alkyl group directly or indirectly bonded to the silicon atom; R₃ is selected from nonhydrolyzable and hydrolyzable groups different from R₂, for instance R₃ may be a nonhydrolyzable group such as alkyl, which may be optionally substituted with epoxy, amino, mercapto, ureido (H₂NC(═O)NH—), or interrupted with one or more sulfur or oxygen atoms, alkenyl, (e.g. vinyl, allyl, methallyl, hexenyl, etc), (alk) acryloxyalkyl (e.g. acryloxypropyl or methacryloxypropyl), and aryl, or R₃ may be a hydrolyzable group such as alkoxy, halogen, acyloxy (e.g. acetoxy, (alk) acryloxy, etc.), hydroxy mercapto, amino or mixtures thereof; R₄ and R₅ are hydrolyzable groups including alkoxy, halogen, acetoxy, hydroxy, and so forth, or mixture thereof; and m and n are each independently 1 to 20.

In another embodiment, the silane copolymer utilized is octyltriethoxysilane/tetraethoxysilicate. In another embodiment, the silane terpolymers comprise the following general structure: R[SiR₁R20]t, 1[SiR₃R₄₀)n-[SiR₆R₇OJpR₅ where R and R₁ are hydrolyzable groups such as alkoxy, halogen, acetoxy, hydroxy, and so forth, or mixture thereof; R₂ is a nonhydrolyzable C₁-C₂₀, aliphatic, cycloaliphatic or aromatic alkyl group directly or indirectly bonded to the silicon atom; R₃ is selected from nonhydrolyzable and hydrolyzable groups different from R₂; R₄ and R₅ are hydrolyzable groups such as alkoxy, halogen, acetoxy, hydroxy or mixtures thereof; R₆ may be a nonhydrolyzable group such as alkyl, vinyl, methacryloxy, or any unsaturated double bond rather than vinyl, or may be a hydrolyzable group such as alkoxy, halogen, acetoxy, hydroxy, and so forth, or mixture thereof; R₇ is a hydrolyzable group such as alkoxy, halogen, acetoxy, hydroxy, and so forth, or mixture thereof; and m, n and p are each independently 1 to 20.

In another embodiment, the invention provides precondensed silane copolymers. In another embodiment, the invention provides single alkylalkoxysilanes. In another embodiment, the invention provides that the copolymers are used in combination with a polysiloxane. Suitable polysiloxanes have the following general formula: (RnS′0(4−n)/2) m wherein R is an organic or inorganic group; n is 0 to 3; and m is equal or greater than 2.

In another embodiment, alkylsilanes comprise alkyltrialkoxysilanes, alkyltrimethoxysilanes and alkylriethoxysilanes including but not limited to methyltrimethoxysilanes, octyltrimethoxysilanes, butyltrimethoxysilanes, dodecyltrimethoxysilanes, octadecyltrimethoxysilanes, methyltriethoxysilanes, octyltriethoxysilanes, butyltriethoxysilanes, dodecyltriethoxysilanes, octadecyltriethoxysilanes, and so forth.

In another embodiment, alkylsilanes and/or alkylsilane copolymers are used in combination with nonhydrolyzable polysiloxanes. In another embodiment, polysiloxanes include the group of triorganosilyl terminated polydiorganosiloxanes including Silwet L-45 polydimethylsiloxane (PDMS), vinyl phenylmethyl terminated dimethyl siloxanes, divinylmethyl terminated PDMS and like, PDMS with polyether pendant groups (Silwet&commat; PA-1), and so forth.

The methods described herein for making the hydrogelss can also be prepared as porous, non-porous, or macroreticular beads of any dimension for use according to the invention. In another embodiment, polymerization may is carried out in the presence of a precipitant, i.e., a liquid which acts as a solvent for the mixture and is chemically inert under the polymerization conditions. In another embodiment, the solvent is present in such amounts as to exert little solvating action.

In another embodiment, the hydrogel matrix elasticity or compliance have a dramatic effect on many aspects of cell function including adhesion, migration, proliferation and differentiation. In many cases, cells appear to have a preferred compliance on which they optimally function. In another embodiment, the hydrogel matrix elasticity or compliance matches well with those of the tissue environment from which they are derived. In another embodiment, the use of hydrogel matrix provides a significant paradigm shift as most of the substrates used thus far, including glass and plastics are orders of magnitude stiffer than the microenvironments found in vivo. In another embodiment, the hydrogel matrix comprises mechanical properties that can be easily tuned for a wide variety of cells. In another embodiment, dextran hydrogels are biocompatible and comprise biomimetic surface that are physicochemically similar to the glycosylated layer found on biological substrates found in vivo. In another embodiment, the invention provides a novel two-component dextran hydrogel with a physiologically relevant range of compliance and the method to covalently immobilize them onto glass substrates.

In another embodiment, the degree of substitution (DS) of the macromonomer is used to form crosslinks. In another embodiment, these groups and the gelation conditions are typically used to control the crosslink density of the resulting hydrogel which in turn determines a wide variety of important properties including the swelling, transport/release kinetics and mechanical properties. In another embodiment, the methods provide immobilized dextran hydrogel system that is capable of achieving a range of physiologically relevant compliances for use as cell-interactive substrates. In another embodiment, intramolecular crosslinking is significant at low macromonomer concentration and hindered the formation of robust hydrogels of low compliance using this method. In another embodiment, the present invention provides a two-component system that combines macromonomers of both high and low degrees of substitution. In another embodiment, a two-component system provides a more uniform control over compliance in the entire range tested. In another embodiment, the present invention provides that immobilized dextran hydrogels are excellent materials on which to build in the biochemical and mechanical cues to develop artificial extracellular matrices for biotechnology and tissue engineering.

In another embodiment, the cells used in the scaffolding comprising the hydrogels described herein and used in the methods provided, are myocyte precursor cells. In another embodiment the cells are cardiac myocytes, or skeletal myocytes, satellite cells, fibroblasts, cardiac fibroblasts, chondrocytes, osteoblasts, endothelial cells, epithelial cells, embryonic stem cells, hematopoetic stem cells, neuronal cells, mesenchymal stem cells, anchorage-dependent cell precursors, or combinations thereof in other discrete embodiments.

In one embodiment, The tissue scaffolding described herein further comprises tissue grown over the scaffolding. In another embodiment provided herein is a method of making an implant for supporting tissue on-growth, the method comprising: providing the three-dimensional tissue scaffold described herein, or a preconditioned artificial tissue comprising living cells that are attached to the three-dimensional tissue scaffold. In one embodiment, provided herein is a method of treatment comprising implanting in the body of a subject artificial tissue made with the preconditioned artificial tissue described herein, wherein in certain embodiment, the living cells used in the scaffolds and methods described herein, originate from the subject receiving treatment.

In one embodiment, a two- or three-dimensional scaffold is provided for ex vivo applications where the scaffold-ECM protein composite allows for the culture of cells which preserves their normal phenotypic structure and function. This refers in another embodiment, to subtle changes in gene and protein expression or more dramatic changes such the formation of multi-cellular structures. The preservation of these cellular characteristics is essential in one embodiment, in order for these cells to be useful for scientific research or diagnostic purposes.

In one embodiment, MCF10A mammary epithelial cells are cultured on the dextran scaffold using the methods described herein, so as to promote the formation of functional multi-cellular structures (“acini”) found in vivo. This would not be possible otherwise without being able to optimize both the mechanical (stiffness, G′<1 kPa) and biochemical (matrix protein=laminin) properties, as in the dextran scaffold. The cells can survive and grow on a wide range of stiffnesses and ECM proteins. In another embodiment, the actini only form these functional structures in culture when the scaffold is tailored specifically.

In one embodiment, the cells do not just survive and grow on the scaffold but to grow correctly so their biological function is “correct”—i.e. as found in vivo. In one embodiment the phenotype of other cells such as endothelial, smooth muscle, osteoblasts and stem cells are also affected by their matrix properties. Accordingly, the scaffolds described herein which are used in the methods provided are optimized in one embodiment for the cell type encapsulated within and is sought to be cultured.

### EXPERIMENTAL DETAILS SECTION

### Materials and Methods

Commercially available dextran from Leuconostoc mesenteroides NRRL B-512(F) was used for this work. Notably, dextran from this source is known to possess low degree of (1→3) branching (5%) and therefore possess more uniform properties. Dextran (MW˜15-20 kDa), Glycidyl methacrylate (GMA), ammonium persulfate (APS), N,N,N′,N′-Tetramethylethylenediamine (TEMED) and all other reagents were purchased from Sigma-Aldrich unless otherwise noted. 4-(dimethylamino)pyridine (DMAP), dimethylsulfoxide (DMSO) and ethanol were purchased from Acros Organics.

### Macromonomer Preparation and Characterization

Dextran macromonomers of varying degrees of substitution (DS) were prepared. 50 g of dextran and 10 g of DMAP were fully dissolved in 450 mL of anhydrous DMSO under argon. Glycidyl methacrylate (7.3 g for DS 1/10; 2.9 g for DS 1/23) was added and the reaction was allowed to proceed for 24 h at room temperature. The reaction mixture was precipitated in excess ethanol and collected/dried by vacuum filtration. The powder was dissolved in ultrapure water (Millipore, Bedford, Mass.) and dialyzed against excess water with 0.02% sodium azide for 36 h using 10 kDa MWCO Snakeskin dialysis membranes (Pierce Biotechnology). The diasylate was lyophilized and stored at −80° C. until use. To verify the modification procedure, Fourier Transform Infrared Spectroscpy was performed on a Nicolet Nexus 470 FT-IR (Thermo Fisher Scientific Inc., Waltham, Mass.) using a sample pressed in a KBr pellet. All chemicals were purchased from Sigma-Aldrich unless otherwise noted.

### Dextran Hydrogels

The macromonomers were dissolved in 0.2 M phosphate buffer solution (pH 8.5) and mixed with 2 mg/ml APS initiator and 5 μl/ml TEMED catalyst to achieve gelation. Unless otherwise noted, the PBS was de-oxygenated by bubbling it with argon for at least 1 h prior to use. For the two-component hydrogels, macromonomer solutions (200 mg/ml) of different degrees of substitution (DS of 1/10 and 1/23) were mixed thoroughly in different ratios prior to the addition of APS and TEMED. The samples were washed with PBS prior to use.

### Immobilized Hydrogel Substrate Preparation (2D).

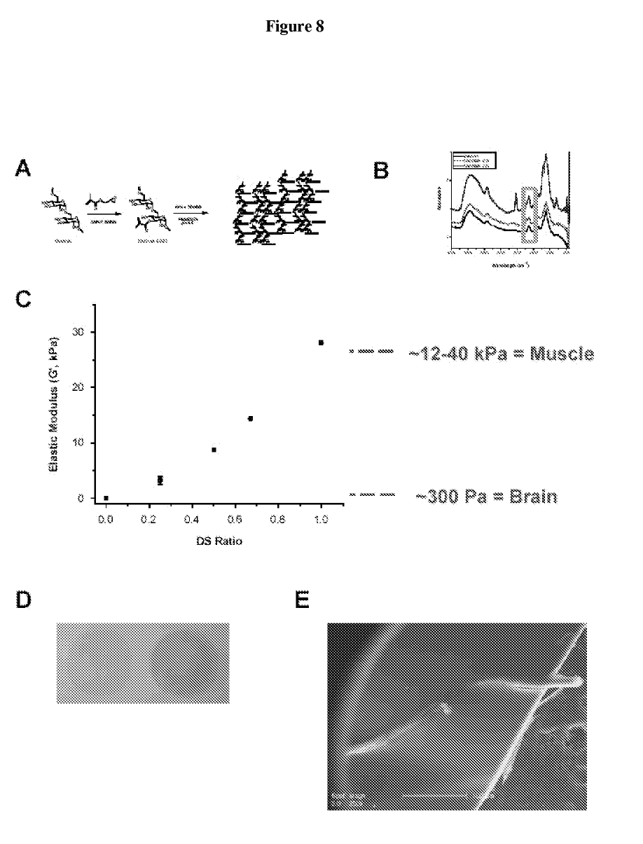

Activated glass coverslips (d=18 mm, 25 mm, Fisher) were prepared using the general silane protocol of Lee et al. (Biomat 2005). Briefly, coverslips were cleaned with piranha solution and washed thoroughly. After drying, the coverslips were immersed in 10% methacryloxypropyltrimethoxysilane in anhydrous toluene for 16 h. After sonication in DMF and ultrapure water, the samples were dried and stored in the dark at 4° C. until use. Immobilized hydrogels were prepared using the following general procedure. Macromonomer solutions (200 mg/ml in 0.2 M PBS, pH 8.5+1 mM EDTA) corresponding to the appropriate mechanical stiffness were prepared using various ratios of DS 1/10:DS 1/23 (see FIG. 8). The macromonomer solution (45 ∝l for 18 mm, 65 ∝l for 25 mm), ammonium persulfate (2 mg/ml, APS) and N,N,N′,N′-Tetramethylethylenediamine (5 ul/ml, TEMED) were mixed and placed onto the activated coverslip. During polymerization, a hydrophobically modified coverslip was placed on top to form a uniform, thin gel. The sample was stored in PBS until use.

### Rheometry

The viscoelastic properties of dextran hydrogels were characterized using an RFS II Rheometer (Rheometrics Inc., Piscataway, N.J.). The standard straincontrolled oscillatory protocol was used (2% strain, 1 rad/s, 1 mm separation distance) for all measurements. The hydrogels were polymerized in situ between 25 mm stainless steel parallel plates during measurement collection and the plateau shear storage modulus (G′) was recorded as the metric for hydrogel stiffness.

### Hydrogel Immobilization

Glass coverslips (d=25 mm, Fisher Scientific) were “activated” and characterized using the general alkylsilane protocol of Lee et al. Briefly, coverslips were cleaned with piranha solution and washed thoroughly. After drying, the coverslips were immersed in 10% methacryloxypropyltrimethoxysilane in anhydrous toluene for 16 h. After sonication in DMF and ultrapure water, the samples were dried and stored at 4° C. until use. Immobilized dextran hydrogels were prepared by combining the reactants in a mold built in-house (FIG. 1). The gel thickness was tightly controlled by using feeler gauges (McMaster-Can, Robbinsville, N.J.) of different sizes as spacers. By using a teflon release surface, gels were removed directly from the mold without the addition of contaminating release agents.

### Atomic Force Microscopy Measurements

Force-displacement spectroscopy of the immobilized dextran hydrogels and ultrathin dextran layers were performed using a Molecular Imaging PicoPlus scanning force microscope (Molecular Imaging Corp, Agilent Technologies). The measurements were made in PBS using Magnetic AC (MAC) Type IV cantilevers having spring constants of 0.05 N/m. Picoscan 5.3.3 were used for analysis of the data.

Functionalization of Hydrogels with ECM Proteins

Aldehyde functional groups were selectively introduced into dextran hydrogels using periodate oxidation. The general protocol, was performed by immersing the hydrogels in sodium periodate solution (13.2 mg/ml in water, pH 5.5) at room temperature for 30 min. The % conversion of the glucopyranoside rings to aldehydes was calculated. For surface-selective ligand immobilization, polydimethylsiloxane (PDMS) stamps were modified with UV/O for 5 min. The periodate solution was then inked and briefly dried before being placed in contact with the hydrogel for 30 min. All samples were washed thoroughly with water and PBS.

The hydrogels were then immersed in 10 mM N- [symbol] (Maleimidocaproic acid) hydrazide (EMCH, Pierce) in 0.1 M PBS (pH 6.5) at 25 C for 4 h. After washing in PBS, the gels were immersed in 100 ∝g/ml fibronectin or laminin in 0.1 M sodium borate buffer (pH 8.5) at 4° C. overnight. The samples were thoroughly washed in 0.1 M PBS and 0.1 M TBS before cell culture experimentation.

### Biodegradation Assay

Dextranase was used to trigger the biodegradation of dexgel matrices. A colorimetric assay using dinitrosalicylic acid (DNSA) to detect isomaltose, the degradation product of dextran, was used. The cultured hydrogel samples were washed with PBS.

### Cell Culture

Human aortic smooth muscle cells (HASMC, Cascade Biologics) were maintained in complete medium consisting of Medium 231 with SMGS supplement using standard cell culture techniques. Cells of low passage (p4-p8) were used for experimentation and serum starved for less than 16 h. Cells were released with 0.05% Trypsin-EDTA for 5 min., neutralized using trypsin inhibitor and resuspended in serum-free medium. Cells were seeded at a density of 50,000 cells/well in 12-well bacteriological plates containing the 18 mm hydrogel samples. The cells were allowed to attach in serum-free medium overnight (12 h); this was replaced with complete medium+100 ∝g/mL penicillin/streptomycin and 100 ∝g/mL gentamycin at day 1. For differentiation assays, the media was replaced with medium 231 supplemented with SMDS+antibiotics. At the appropriate time point, the cells were washed with PBS and fixed with 3.7% formaldehyde solution for subsequent spreading analysis or immunostaining procedure. HT1080 fibroblasts were cultured similarly with the exception of the culturing medium which was DMEM+10% FBS.

Encapsulation of Cells with Hydrogels (3D)

A polymerizable ECM crosslinker for 3D hydrogels was synthesized by conjugating various ECM proteins to an acrylate functional crosslinker. 1-2 mg/ml Fibronectin (Invitrogen, Fn), Laminin (Invitrogen, Lm) or Bovine Serum Albumin (Fisher, BSA) were reacted with 100 fold molar excess ACRL-PEG-NHS 3400 crosslinker (Nektar Therapeutics) in PBS, pH 7.4 at 4 C for 4 h. Sterile dextran macromonomer, APS and TEMED were mixed with 10% (by vol.) ECM crosslinker solution and used to resuspend pelleted cells at a density of 1-2×10₆ cells/ml. All other culturing procedures were the same as the 2D gel experiments. For Live/Dead staining, calcein and ethidium homodimer in PBS+4.5 g/L glucose were used to fluorescently label cells prior to imaging using a Zeiss LS 510 confocal microscope.

### Assays for Cell Function

Cells spreading was observed via phase contrast microscopy and the area was quantified as a function of modulus for all cells. To determine the differentiative status of HASMC cells on the gels, [symbol] -smooth muscle actin (clone 1A4), calponin (clone hHCD) and caldesmon (hCP) were selectively stained. F-actin (Phalloidin) and nuclei (DAPI) were also stained to determine co-localization in some cases.

### Example 1

### Dex-GMA Macromonomers

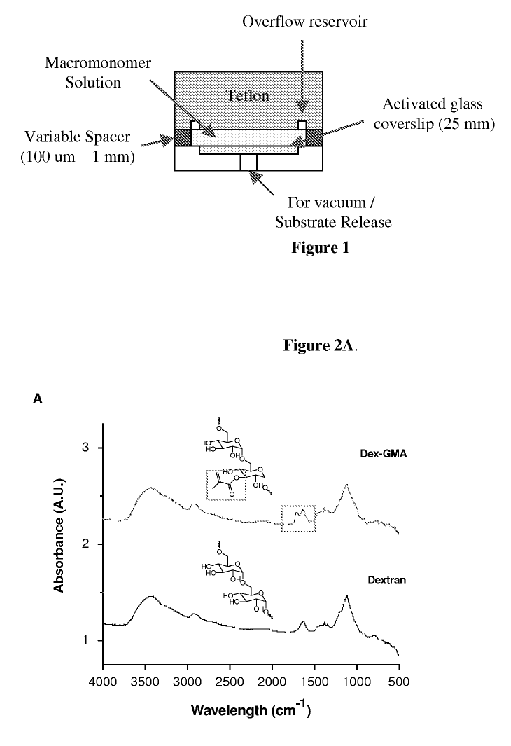

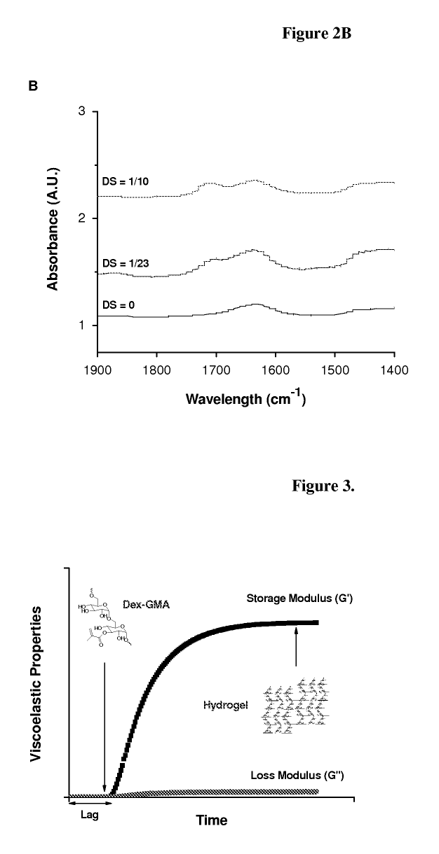

Macromonomers were synthesized by modifying the free hydroxyl groups with glycidylmethacrylate (GMA). The incorporation of methacrylate units was verified using FTIR. FIG. 2a shows the FTIR spectra of the modified and unmodified dextran which shows the increase of peaks at 1710 cm⁻¹ corresponding to the methacrylate carbonyl groups.(van Dijk-Wolthuis et al. 6317-22) The ratio of the peak intensities at 1710 cm⁻¹ and 1640 cm⁻¹ was calculated to compare the Dex-GMA macromonomers of different degrees of substitution (DS): DS 0=0.18; DS 1/23=0.64; DS 1/10=0.77. The peak at 1640 cm⁻¹ is a peak inherently present in dextran and was used as an internal control to account for variations in sample amounts. The increased incorporation of functional methacrylate units to the hydroxyl groups of dextran and the ability to control the DS by reaction conditions were therefore both verified. The DS values used in the manuscript are based on those verified previously by NMR with a similar experimental system.

The mechanical properties of the gel were controlled by varying degrees of substitution (DS) and the macromonomer concentration [FIG. 8B]. Optimal control over the full range of hydrogel compliance (G′˜0.4 to 42 KPa) was achieved by using a blend of two macromonomers of different DS [FIG. 8C]. The physical integrity and homogeneity of dextran hydrogels were verified at the macroscopic by Alcine Blue staining [FIG. 8D] and microscopic level by environmental SEM [FIG. 8E]. In the eSEM image, a part near the edge of the gel was imaged to provide contrast; the hydrogel was highly hydrated and homogeneously featureless at this scale.

### Example 2

### Gelation of Dextran-GMA Macromonomers

The macromonomers were polymerized radically using APS initiator and TEMED catalyst and tracked in situ by rheometry. FIG. 3 shows a representative data set. After a short lag period, the elastic component of the modulus (Storage Modulus, G′) increased sharply as gelation occurred. The viscous component (Loss Modulus, G″) increased negligibly during this time for all hydrogels and therefore is not reported in subsequent figures. As the plateau in G′ represents completion of gelation, the steady-state value was used as the metric for hydrogel compliance.

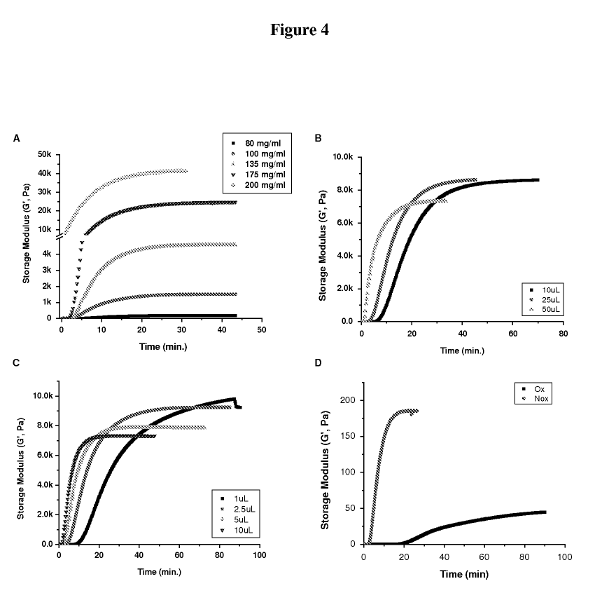

The effect of various experimental parameters that control macromonomer gelation was investigated in order to optimize the system. FIG. 4 shows the effect of (A) macromonomer concentration, (B) initiator concentration, (C) catalyst concentration and (D) medium oxygen content on the kinetics of gelation and hydrogel compliance. Dex-GMA concentration was the most effective parameter to precisely control hydrogel compliance in the physiologically relevant range of 400 Pa (80 mg/ml) to 42 KPa (200 mg/ml). Varying the initiator and catalyst concentrations altered the kinetics of gelation but did not significantly alter hydrogel compliance. The oxygen content in the buffer used to dissolve the reactants had a significant inhibitory effect on gelation, increasing the lag time and decreasing both the reaction rate and hydrogel compliance. Presumbly, this can be attributed to the quenching effect that oxygen has on radical lifetime. This effect was more significant at lower Dex-GMA concentrations and lower compliance. Displacement of the oxygen in the buffer with argon bubbling for at least one hour (nox) was sufficient to inhibit the qunching reaction.

### Example 3

### One-Component Vs. Two-Component Hydrogels

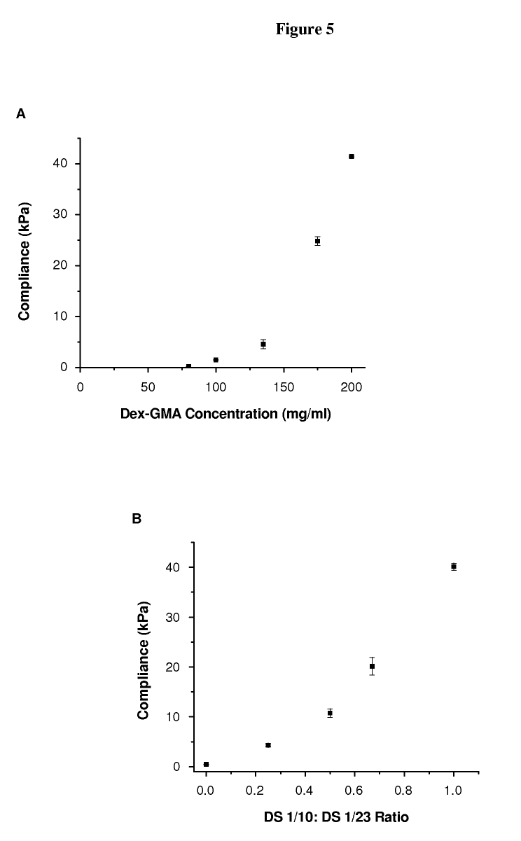

One drawback of the dextran macromonomer/hydrogel system described thus far is that the amount of dextran in the final hydrogel varies with time. In addition, hydrogels prepared at low macromonomer concentrations (<100 mg/ml) were fragile which made the low compliance hydrogels difficult to manipulate. A two-component dextran hydrogel system using mixtures of macromonomers of different degrees of substitution (DS of 1/10 and 1/23) was developed to address these issues. FIG. 5A and FIG. 5B shows the compliance of hydrogels made using the one-component and two-component hydrogels respectively. The same compliance range (G′˜400 Pa−42 KPa) was achieved by both systems however the compliance of the two-component system varied more uniformly in the entire range.

### Example 4

### Novel Activated Substrates for Hydrogel Immobilization

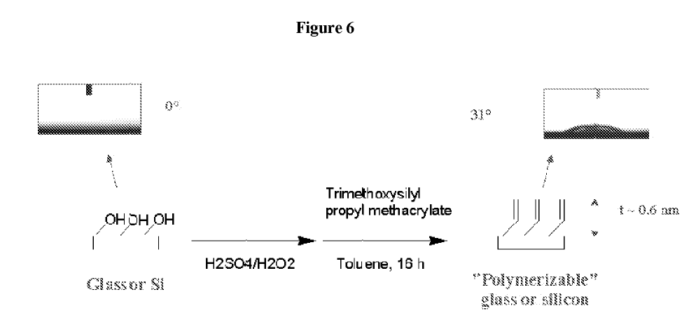

Many biotechnological applications, such as cell culture, require that the hydrogels be immobilized to substrates to provide stability during manipulation and experimentation. Flexible surface modification method based on ω-functional alkylsilanes were used to modify glass coverslips which can covalently crosslink into the hydrogel. The reaction scheme and the properties of the ultrathin methacrylate film are presented in FIG. 6. The increases in the thickness of 0.6 nm and in the water contact angle of 31° confirmed surface activation. When the macromonomer was gelled in contact with the activated substrates using the mold shown in FIG. 1, they were more stable to both mechanical manipulation and extended immersion in aqueous conditions.

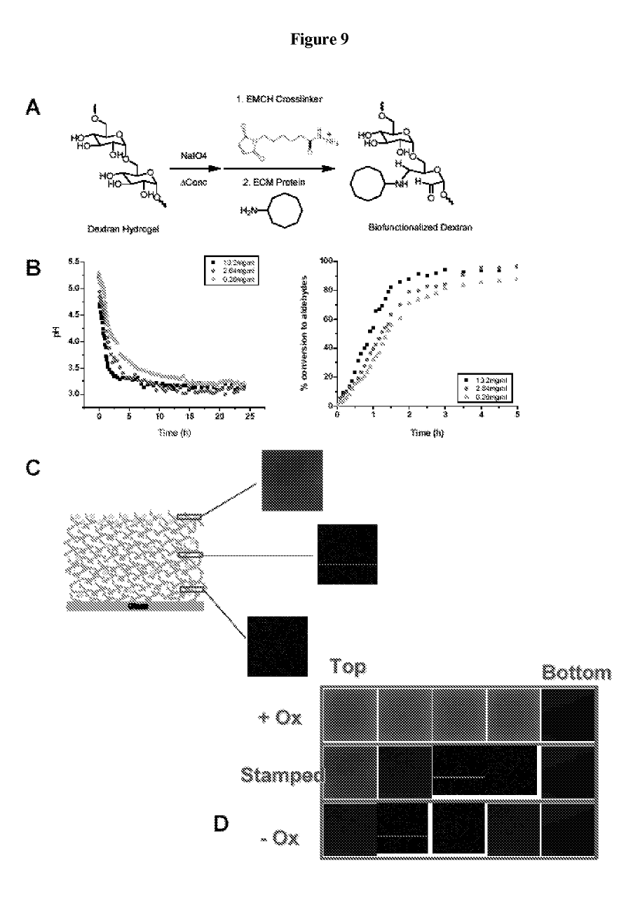

Fine control over both the amount and spatial location of ECM ligand attachment to dextran gels was possible at the material level through the use of selective periodate oxidation and a hydrazide-based crosslinker [FIG. 9A]. Periodate concentration was used to sensitive control the amount of aldehyde groups generated for subsequent attachment [FIG. 9B]. In order to selectively modify only the surface of the hydrogels [FIG. 9C], stamps made of UV-treated Polydimethylsiloxane (PDMS) stamps were used to localize the periodate oxidation. After immersion in a solution of AlexaFluor 488-labeled fibronectin, the ligand concentration was selectively enhanced at the surface [FIG. 9D]. By comparison, the fully oxidized gel (by immersion) was homogeneously fluorescent and the non-oxidized hydrogels were homogeneously unmodified

### Example 5

### Stiffness of Immobilized Hydrogels Vs. Thin Dextran Films

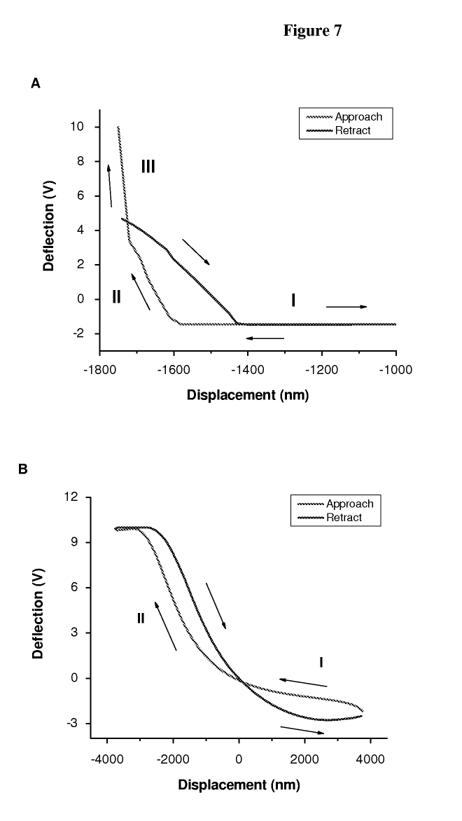

Stiffness assessment of two types of highly hydrated biomimetic substrates using in situ techniques such as AFM was performed. The results of the force spectroscopy of a dextran film of 120 nm thickness (hydrated) and a 42 KPa dextran hydrogel of 500 μm thickness (hydrated) are shown as FIGS. 7A and 7B respectively. The results from dextran films show clear transitions as the tip traveled from I) the buffer to II) the hydrated layer and III) the glass substrate underneath. The slopes of region II and III correspond to the compression of the hydrogel and the bending of the cantilever respectively. Therefore, the slope in region III was calibrated to the cantilever spring constant and the slopes of the region II of the two figures were used to compare material stiffnesses. The stiffest dextran hydrogel used herein (G′=42 KPa) was found to be over 100 times softer than the relatively thick dextran film.

### Example 6

### Cell Culture with Dextran Hydrogels

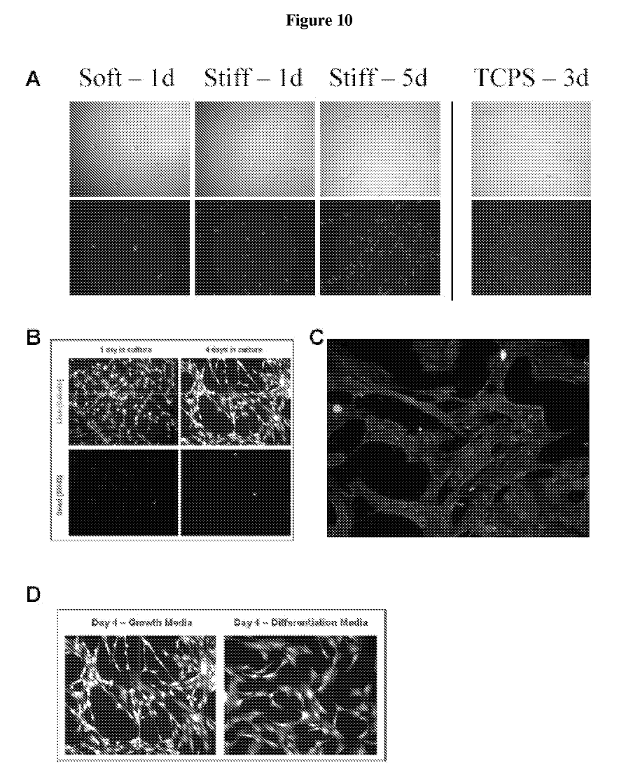

The effect of hydrogel compliance on human aortic smooth muscle cell (HASMC) function was assessed by spreading [FIG. 10A] and live/dead [FIG. 10B] assays at 1 and 5 days. The hydrogels were immobilized on glass coverslips and functionalized with fibronectin (Fn) prior to cell seeding. After 1 day, HASMCs remained round on “soft” (1 KPa) gels whereas the cells were spread on “stiff” (40 KPa) gels. Cells on TCPS substrates after 3 days are provided as a positive control. Cells on both substrate types were viable at 1 day. After 5 days, the cells on stiff substrates proliferated to achieve a greater surface coverage while no viable cells were found on the soft substrate. By day 5, the HASMCs on stiff dextran substrates organized into multi-cellular structures with significant stress fibers [FIG. 10C—nuclear staining with DAPI is in blue; F-actin staining with phalloidin is in red]. The differentiative capability of HASMCs cultured on stiff dextran substrates was maintained and demonstrated by culturing the cells with differentiation media for 4 days. [FIG. 10D] The appearance of the cells were of the less contracile phenotype represented by the larger, stellate morphology of the calcein labeled cells.

3D Cell Culture with Dextran Hydrogels

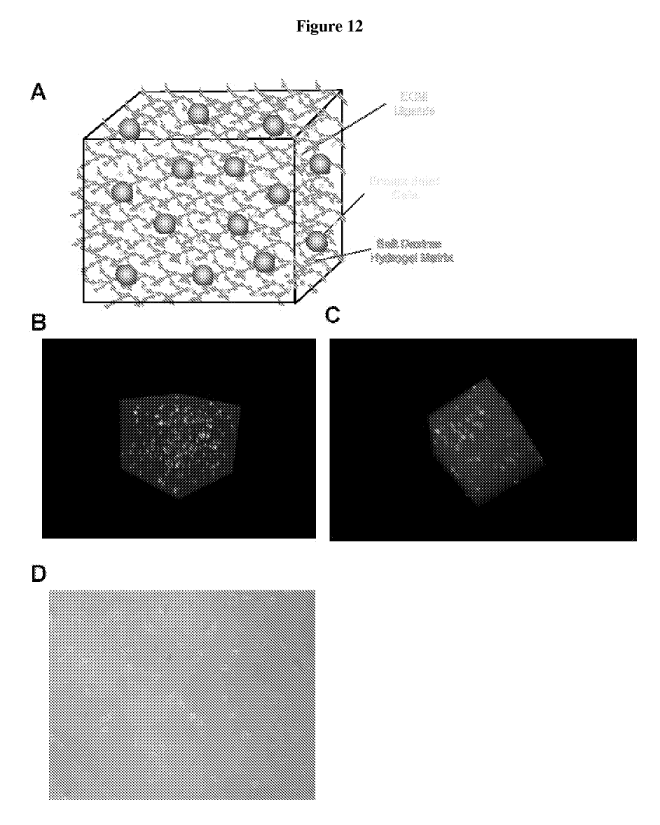

HT1080 cells were encapsulated into the dextran hydrogels in situ by resuspending the cells in the macromonomer solution prior to gelation [FIG. 12A]. Acrylate-based crosslinker was functionalized first with either BSA or fibronectin (Fn) prior to co-polymerization with the macromonomer. Confocal fluorescent microscopy was used to analyze the viability of the encapsulated cells in the three dimensional dextran matrices with BSA [FIG. 12B] and with Fn [FIG. 12C] after two days. In the assay, green and red labeling represent live (calcein) and dead (EthD) cells respectively. As the goal of the three-dimensional culture system was to be able to recover the functional cells, the matrix was degraded using 20 U of dextranase at PBS+dextrose pH 6.0, 2 mM MnCl2, at 37° C. The supernatant was collected and placed onto a glass substrate coating with 5 mg/ml fibronectin. After 2 h, the media was gently changed to growth media. After 24 h, phase contrast micrographs after 24 h showed that the recovered cells appeared normal in appearance and were spreading normally on the Fn-coated surfaces.

### Example 7

### Cell Recovery by Biodegradation of Dextran Hydrogels

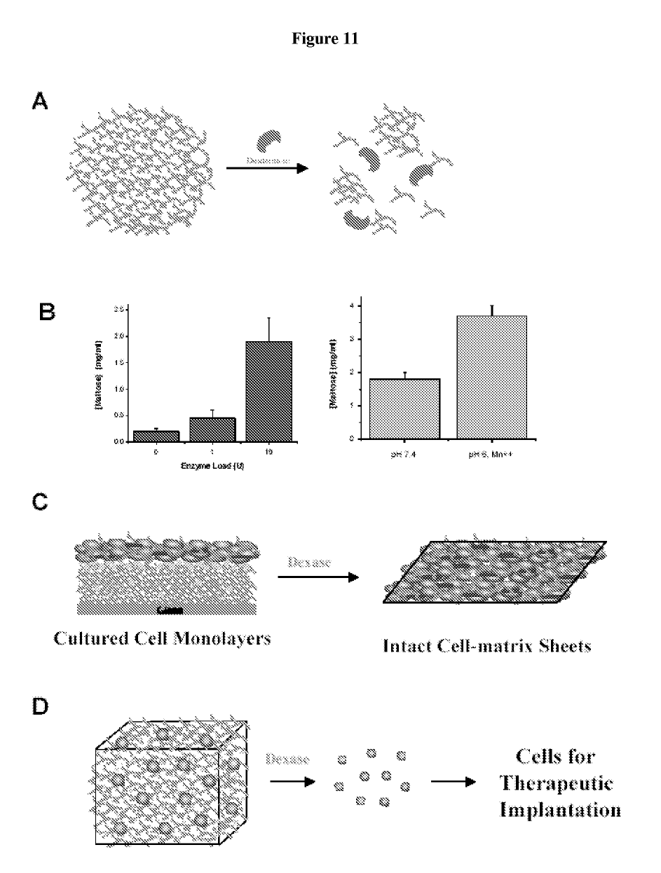

The conditions of the enzymatic degradation of dextran hydrogels by dextranase [FIG. 11A] were optimized for use with dextran hydrogels. By using a colorimetric assay to monitor the degradative products of the hydrogel after 16 h exposure, the enzyme load, pH and Mn²⁺ ion concentration were the key parameters controlling this reaction [FIG. 11B]. There are two ways in which the biodegradation of dextran can be of use in cell culture: The release of monolayers of 2D cultured cells [FIG. 11C] and the release of cells from three dimensional cultures of cells [FIG. 11D] without disruption of the deposited matrix or of the functional multi-cellular units formed by the cells.

What is claimed:

1. A composition comprising a copolymer comprised of a first and a second dextran macromonomer wherein each of said first and second dextran macromonomer comprises a different degree of glycidylmethacrylate (GMA) substitution.

2. The composition of claim 1, wherein the degree of substitution in the first macromonomer is between about 1/23 and 1/10.

3. The composition of claim 1, wherein the degree of substitution in the second macromonomer is between about 1/23 and 1/10.

4. The composition of claim 1, wherein said substitution is a methacrylate-hydroxyl substitution, or a carbonate-hydroxyl substitution.

5. The composition of claim 1, further comprising an extra cellular matrix protein covalently bound to said dextran macromonomer.

6. The composition of claim 5, wherein said extra cellular matrix protein is laminin, fibronectin, collagen or their combination.

7. A biomimetic hydrogel comprising a copolymer having a first and a second dextran macromonomer wherein each said dextran macromonomer comprises a different degree of glycidylmethacrylate (GMA) substitution; and crosslinking agent.

8. The hydrogel of claim 7, wherein the degree of substitution in the first macromonomer is between about 1/23 and 1/10.

9. The hydrogel of claim 7, wherein the degree of substitution in the second macromonomer is between about 1/23 and 1/10.

10. The hydrogel of claim 7, wherein the substitution is a methacrylate-hydroxyl substitution, or a carbonate-hydroxyl substitution.

11. The hydrogel of claim 7, further comprising a covalently bound extra cellular matrix protein to the dextran macromonomer.

12. The hydrogel of claim 11, wherein the extra cellular matrix protein is laminin, fibronectin, collagen or their combination.

13. The hydrogel of claim 7, wherein the storage modulus (G′) is between about 400 pa to 42 Kpa.

14. The hydrogel of claim 7, wherein the catalyst is TEMED, DEMED or their combination.

15. The hydrogel of claim 14, wherein further comprising ammonium persulfate (APS), or riboflavin.

16. A three-dimensional tissue scaffold for supporting tissue on-growth, the scaffold comprising: a substrate immobilized hydrogel, wherein the hydrogel comprises a copolymer having a first and a second dextran macromonomer wherein each of said first and second dextran macromonomer comprises a different degree of glycidylmethacrylate (GMA) substitution; and at least one of a living cell, an ECM ligand, protein, peptide, transcript factor, cytokine, therapeutic agent, growth factor, encapsulated in the hydrogel or on its surface.

17. The tissue scaffolding of claim 16, wherein said substrate comprises cobalt, silicon, plastic, chromium, glass, stainless steel, polystyrene, tantalum, titanium, carbon, calcium, quartz, ceramic, or a combination thereof.

18. The tissue scaffolding of claim 16, wherein the cells are myocyte precursor cells, cardiac myocytes, skeletal myocytes, satellite cells, fibroblasts, cardiac fibroblasts, chondrocytes, osteoblasts, endothelial cells, epithelial cells, embryonic stem cells, hematopoetic stem cells, neuronal cells, mesenchymal stem cells, anchorage-dependent cell precursors, or combinations thereof.

19. The tissue scaffolding of claim 16, further comprising tissue grown over the scaffolding.

20. A method of making an implant for supporting tissue on-growth, the method comprising: providing the three-dimensional tissue scaffold of claim 16.

21. A preconditioned artificial tissue comprising living cells that are attached to the three-dimensional tissue scaffold of claim 16.

22. The scaffold of claim 16, wherein the degree of substitution in the first macromonomer is between about 1/23 and 1/10.

23. The scaffold of claim 16, wherein the degree of substitution in the second macromonomer is between about 1/23 and 1/10.

24. The scaffold of claim 16, wherein the substitution is a methacrylate-hydroxyl substitution, or a carbonate-hydroxyl substitution.

25. The scaffold of claim 16, further comprising a covalently bound extra cellular matrix protein to the dextran macromonomer.

26. The scaffold of claim 26, wherein the extra cellular matrix protein is laminin, fibronectin, collagen or their combination.

27. The scaffold of claim 16, wherein the storage modulus (G′) is between about 400 pa to 42 Kpa.

28. The scaffold of claim 27, whereby the storage modulus is no more than 1 Kpa.

29. The scaffold of claim 16, whereby the living cell maintains its phenotypic structure.

30. An implant comprising the scaffold of claim 16.

31. A method of modulating the amount and location of an ECM ligand attachment on the surface of a hydrogel comprising the step of: selectively modifying the aldehyde concentration and location at the surface of a hydrogel comprising a first and a second dextran macromonomer wherein each of said first and second dextran macromonomer comprises a different degree of glycidylmethacrylate (GMA) substitution; and covalently binding the ECM ligand to the aldehyde group at the surface of the hydrogel.

32. The method of claim 31, whereby the step of selectively modifying the aldehyde concentration at the surface of the hydrogel comprises exposing only the surface of the hydrogel to periodate oxidation.

33. The method of claim 31, whereby the periodic acid is immobilized on a substrate.

34. The method of claim 31, whereby the step of covalently binding the ECM ligand to the aldehyde group at the surface of the hydrogel comprises the step of contacting the surface modified hydrogel with the ECM ligand

35. The method of claim 31, whereby the ECM ligand is laminin, fibronectin, collagen or their combination.

36. The method of claim 31, whereby the degree of substitution in the first macromonomer is between about 1/23 and 1/10.

37. The method of claim 31, whereby the degree of substitution in the second macromonomer is between about 1/23 and 1/10.

38. The method of claim 31, whereby the substitution is a methacrylate-hydroxyl substitution, a carbonate-hydroxyl substitution, or a mixture thereof.

39. The method of claim 31, whereby said substrate comprises cobalt, silicon, plastic, chromium, glass, stainless steel, polystyrene, tantalum, titanium, carbon, calcium, quartz, ceramic, or a combination thereof.

40. A method of culturing living cells in a hydrogel while maintaining their phenotypic structure, comprising the steps of: encapsulating the cells in a hydrogel comprising a copolymer comprised of a first and a second dextran macromonomer wherein each of said first and second dextran macromonomer comprises a different degree of glycidylmethacrylate (GMA) substitution; modulating the storage modulus of the hydrogel; covalently binding the hydrogel to an ECM ligand; and allowing the cells to grow.

41. A method of controlling cell culture proliferation on a hydrogel surface, comprising the steps of immobilizing a hydrogel comprising a copolymer of a first and a second dextran macromonomer wherein each of said first and second dextran macromonomers comprises a different degree of glycidylmethacrylate (GMA) substitution, onto a substrate; modulating the storage modulus of the immobilized hydrogel; functionalizing the surface of the hydrogel with an ECM ligand; and seeding the hydrogel with the cell culture whose proliferation is sought to be controlled, wherein below a threshold storage modulus, no cell proliferation will occur.

42. The method of claim 40, whereby each of the first and second macromonomer in the copolymer has a degree of substitution of between about 1/23 and 1/10.

43. The method of claim 42, whereby the substituted dextran macromonomer is a methacrylate-hydroxyl substituted dextran macromonomer, a carbonate-hydroxyl substituted dextran macromonomer, or a mixture thereof.

44. The method of claim 42, whereby the step of immobilizing the hydrogel onto a substrate comprises the steps of:
(a) activating the substrate;
(b) immersing the activated substrate in an agent capable of forming a covalent bond between said activated substrate and said composition thereby making a functionalized substrate;
(c) drying said functionalized substrate, and
(d) contacting the functionalized substrate with the hydrogel

45. The method of claim 42, whereby said substrate comprises cobalt, silicon, plastic, chromium, glass, stainless steel, polystyrene, tantalum, titanium, carbon, calcium, quartz, ceramic, or a combination thereof.
